(19) United States
(12) Patent Application Publication
(10) Pub. No.: US 2006/0228386 A1
(43) Pub. Date: Oct. 12, 2006

(54) Polymeric microstructures

(75) Inventors: Chris Stephens, Knoxville, TN (US); Peter Gregory Engleman, Knoxville, TN (US); Roberto Benson, Knoxville, TN (US)

Correspondence Address: RUDEN, MCCLOSKY, SMITH, SCHUSTER & RUSSELL, P.A., 222 LAKEVIEW AVE, SUITE 800, WEST PALM BEACH, FL, 33401-6112, US

(73) Assignee: University of Tennessee Research Foundation

(21) Appl. No.: 11/359,695

(22) Filed: Feb. 22, 2006

Related U.S. Application Data
(60) Provisional application No. 60/655,177, filed on Feb. 22, 2005.
(60) Provisional application No. 60/715,416, filed on Sep. 9, 2005.

(51) Int. Cl. A61K 8/02

(52) U.S. Cl. 424/401

(57) ABSTRACT

Methods for producing and using polymeric microstructures having a pre-determined geometry (e.g., rectangular prism, cube), and pre-determined surface characteristics are disclosed herein. The polymeric microstructures described herein are particularly useful as microcarriers in cell culture applications because they provide high surface areas, and improved surface/volume ratios over currently available microstructures, and can be manufactured to have pre-determined physiochemical characteristics (e.g., substrate curvature, texture, shape, porosity, surface chemistry) to optimize compatibility with a pre-determined type of cell (e.g., bacterial, animal, mammalian, human) to be cultured. The polymeric microstructures described herein are also particularly useful in tissue engineering (e.g., bone engineering) applications.

### CROSS-REFERENCE TO RELATED APPLICATIONS

This application claims priority from U.S. provisional patent application Ser. No. 60/655,177 filed Feb. 22, 2005, and U.S. provisional patent application Ser. No. 60/715,416 filed Sep. 9, 2005.

### FIELD OF THE INVENTION

The invention relates generally to the fields of polymer science and microstructure technology. More particularly, the invention relates to polymeric microstructures having a particular geometry (e.g., a regular or irregular polyhedron such as a rectangular prism or a cube), and particular surface characteristics (e.g., texture, topology, chemistry); and methods for producing and using such microstructures.

### BACKGROUND OF THE INVENTION

Large-scale cell culture typically involves using a bioreactor to grow cells in suspension in a liquid medium. To facilitate growth of anchorage-dependent cells in a bioreactor, microstructures can be added to provide a substrate to which the cells can adhere. Conventionally, spherical micro-porous beads made by an immiscible liquid-liquid polymerization method (R. Arshady, Review: Beaded Polymer Supports and Gels: I. Manufacturing Techniques, Journal of Chromatography, 1991, 181-197) have been used for this purpose. While useful, because they have a relatively small surface area/volume ratio, spherical beads reduce the efficiency of bioreactor-based cell cultures because they displace a significant portion of a bioreactor's capacity. In addition, surface texture and composition of conventional spherical micro-porous beads made by the immiscible liquid-liquid polymerization method is limited and often less than ideal for cell culture applications. While some attempts at coating the surface of spherical beads have been attempted, these have so far proven to be time-consuming, expensive, and not completely effective.

The ability to control the geometry and surface characteristics (e.g., composition, texture, chemistry, topology) of cell culture microstructures is important for optimizing cell growth conditions as the manner in which a cell interacts with a substrate can have marked effects on its physiology. See, e.g, Hogan et al., Journal of Electron Microscopy Technique 18:106-116, 1991; and Dalby et al., Biomaterials 25:77-83, 2004. Heretofore, only a limited number of different cell culture microstructures have been developed because practical methods for controlling microstructure geometry and surface characteristics have not been available.

### SUMMARY

The invention is based on the development of new methods for making microstructures with pre-determined geometries and surface characteristics. These methods involve synthesizing polymeric microstructures using chain-growth polymerization techniques (e.g., Polymerization Induced Phase Separation (PIPS) and block copolymer methods) within a confined space defining the pre-determined geometry. The surface characteristics of microstructures produced by the chain-growth polymerization methods described herein are controlled by phase separation (e.g. controlled by chain length in PIPS and controlled by block length and solubility parameters in block copolymers). Because chain-growth polymerization is typically a single-step process, the methods of the invention are comparatively cost-effective as problems with manufacturing and handling are limited.

The methods of the invention allow the characteristics of the polymeric microstructures to be manipulated so that microstructures can be custom-designed for particular applications. For instance, the methods allow microstructures to be made that can be used as substrates for cells to adhere to (e.g., microcarriers) in vitro and that are optimal for a specific cell culture method, e.g., those with high surface area to volume ratios and desirable physiochemical characteristics (e.g., optimal substrate curvature, texture, shape, porosity, surface chemistry for a given cell culture application). These advantages associated with surface area and size increase the efficiency of bioreactor-based cell culture systems and may reduce the costs associated with pharmaceutical production by allowing for smaller bioreactors or higher density cell growth in the same size bioreactor. Because the microstructures described herein have a significantly reduced surface area and volume compared to currently available spherical microstructures, they contribute less to the amount of synthetic material present in a culture system. This decrease in synthetic material in a cellular aggregate can improve the accuracy of bioreactor-based disease models, which can reduce the need for animal-based testing in the initial stages of pharmaceutical development. The methods of the invention also allow microstructures to be made that are useful in tissue engineering applications, since the created polymer has a controllable shape, surface texture, and controlled degradability.

Accordingly, the invention features a method for producing a polymeric microstructure having a pre-determined geometric shape. This method includes the steps of: (a) providing a solid support having at least one channel or cavity therein, the at least one channel or cavity defining at least a portion of the pre-determined geometric shape; (b) applying a prepolymer mixture to the at least one channel or cavity; (c) polymerizing the prepolymer mixture in the at least one channel or cavity to result in the formation of the polymeric microstructure having the pre-determined geometric shape, and (d) removing the polymeric microstructure from the solid support. In this method, the solid support can be, for example, a mold, a capillary, a slide, a pair of slides, and a device having at least one microfluidic channel. Step (c) of this method can be performed by techniques including PIPS and polymerizing macromers to create block copolymers. This step includes subjecting the prepolymer mixture to a sufficient amount of irradiation (e.g., ultraviolet light) to polymerize the prepolymer mixture. In some embodiments, this method further includes using a mask to block the irradiation from at least one discrete portion of the prepolymer mixture in the at least one channel or capillary. Step (d) of this method can include applying pressure to the polymeric microstructure within the at least one channel or cavity. The prepolymer mixture can include at least one hydrophobic monomer and at least one hydrophilic monomer. (e.g., Ethylene Glycol Dimethacrylate-co-Trimethylolpropane Triacrylate and Triethylolpropane Triacrylate).

In another aspect, the invention features a polymeric microstructure having a pre-determined geometry, the polymeric microstructure formed from the process including the steps of: (a) providing a solid support having at least one channel or cavity therein, the at least one channel or cavity defining at least a portion of the pre-determined geometric shape; (b) applying a prepolymer mixture to the at least one channel or cavity; (c) polymerizing the prepolymer mixture in the at least one channel or cavity to result in the formation of the polymeric microstructure having the pre-determined geometric shape, and (d) removing the polymeric microstructure from the solid support. For example, polymeric structures having a size between about 30 and 500 microns; those having a shape of a regular polyhedron; those having a shape of an irregular polyhedron; those having a shape of a cube, prism, pyramid, sphere, tetrahedron, octahedron, dodecahedron, rhombic dodecahedron, trapezohedron, icosahedron, or stellated polyhedron; those that are fibers; those that are porous; those that are linked to at least one functional group including a peptide, a protein, an antibody, a saccharide, an organic molecule with a molecular weight of less than 10,000 daltons, and a nucleic acid; those having a surface area in the range of about 5400 μm² to about 1.5×10⁶ μm², a volume in the range of about 27000 μm³ to about 1.25×10⁸ μm³, and a surface area to volume ratio in the range of about 0.2 μm⁻¹ to about 0.012 μm⁻¹; and those including a biodegradable polymer are featured in the invention.

Unless otherwise defined, all technical terms used herein have the same meaning as commonly understood by one of ordinary skill in the art to which this invention belongs.

As used herein, in general the word “microstructure” means a particle having a size between about 1 and 1000 (e.g., between about 10 and 900, 50 and 850, 100 and 700, 150 and 650, 200 and 600, 250 and 550, 300 and 500, 350 and 450, etc.) microns. Similarly, by the term “microstructures” is meant a plurality of particles having an average size of between about 1 and about 1000 (e.g., between about 10 and 900, 50 and 850, 100 and 700, 150 and 650, 200 and 600, 250 and 550, 300 and 500, 350 and 450, etc.) microns. When referring to a microstructure that is a fiber, the term microstructure means a fiber having a second largest dimension between about 1 and about 1000 (e.g., between about 10 and 900, 50 and 850, 100 and 700, 150 and 650, 200 and 600, 250 and 550, 300 and 500, 350 and 450, etc.) microns.

By reference to the “size” of a microstructure is meant the length of the largest straight dimension of the microstructure. For example, the size of a perfectly spherical microstructure is its diameter.

As used herein, the phrase “functional group” means a chemical group that imparts a particular function to an article (e.g., microstructure) bearing the chemical group. For example, functional groups can include substances such as peptides, proteins, oligosaccharides, antibodies, oligonucleotides, biotin, or streptavidin that are known to bind particular molecules; or small chemical groups such as amines, carboxylates, and the like.

Although methods and materials similar or equivalent to those described herein can be used in the practice or testing of the present invention, suitable methods and materials are described below. All publications mentioned herein are incorporated by reference in their entirety. In the case of conflict, the present specification, including definitions will control. In addition, the predetermined embodiments discussed below are illustrative only and not intended to be limiting.

### BRIEF DESCRIPTION OF THE DRAWINGS

FIG. 1 is a schematic illustration of a microfluidic chip design used to create bone tissue engineering carriers.

FIGS. 2A and 2B are schematic illustrations of Polymicro™ square ID capillary tubing.

FIG. 3 is a schematic illustration of a capillary-based synthesis assembly.

FIG. 4 is a series of scanning electron micrograph (SEM) images of a square polymeric fiber corner (left), zoomed in view of corner (middle), and fiber surface (right).

### DETAILED DESCRIPTION

The invention encompasses compositions and methods relating to polymeric microstructures having a pre-determined geometry and pre-determined surface characteristics. The polymeric microstructures can be used in a number of applications, including cell culturing, bone tissue engineering, and other forms of tissue engineering. Methods for producing the polymeric microstructures by polymerization in a confined space defining a particular geometry to result in microstructures having that geometry are described herein. The surface characteristics of such microstructures can be controlled by choosing the particular monomers and solvents used to make the polymer.

The below described preferred embodiments illustrate adaptation of these compositions and methods. Nonetheless, from the description of these embodiments, other aspects of the invention can be made and/or practiced based on the description provided below.

### Polymer Chemistry, Microfluidics, and Cell Culture Methods

Methods involving conventional polymer chemistry and cell culture techniques are described herein. Such techniques are generally known in the art and are described in detail in methodology treatises. Polymer chemistry techniques including chain-growth polymerization are generally known in the art and are described in detail in Polymer Science and Technology by Joel Fried, Prentice Hall, 2^{nd} edition, 2003; Svec and Frechet, Industrial Engineering Chemical Research, 28:34-48, 1999; and Yu et al., Journal of Polymer Science: Part A, 40:755-769, 2002. PIPS techniques are well known in the art and are described in Yu et al., Journal of Polymer Science: Part A, 40:755-769, 2002; Viklund et al., Macromolecules 34:4361-4369, 2001; Viklund et al., Chemistry of Materials 8:744-750, 1996; and Ngola et al., Analytical. Chemistry 73:849-856, 2001. Microfluidic concepts and applications are described in Kamholz et al., Analytical Chemistry, 71:5340-5347, 1999; Kamholz et al., Biophysical. Journal 80:1967-1972, 2001; Yang et al., Analytical Chemistry, 73:165-169, 2001; Chiu et al., Proc. Natl. Acad Sci. USA, 97:2408-2413, 2000; and Dertinger et al., Analytical Chemistry, 73:1240-1246, 2001. Cell culture techniques are also generally known in the art and are described in detail in as Culture of Animal Cells: A Manual of Basic Technique, 4^{th} edition, by R. Ian Freshney, Wiley-Liss, Hoboken, N.J., 2000; General Techniques of Cell Culture, by Maureen A. Harrison and Ian F. Rae, Cambridge University Press, Cambridge, UK, 1994; and Hu and Aunin, Curr Opin Biotechnol. 8:148-153, 1997.

### Methods for Producing Microstructures

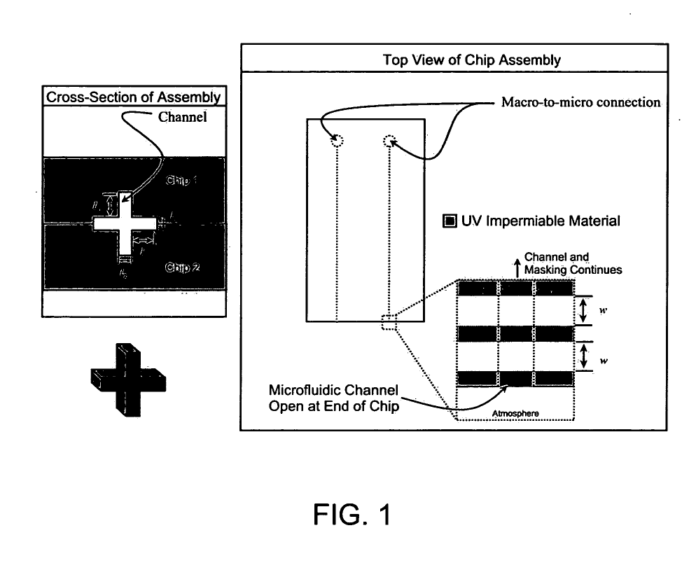

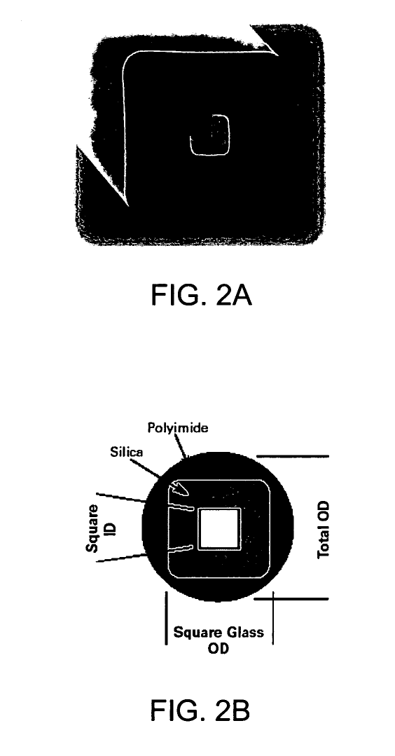

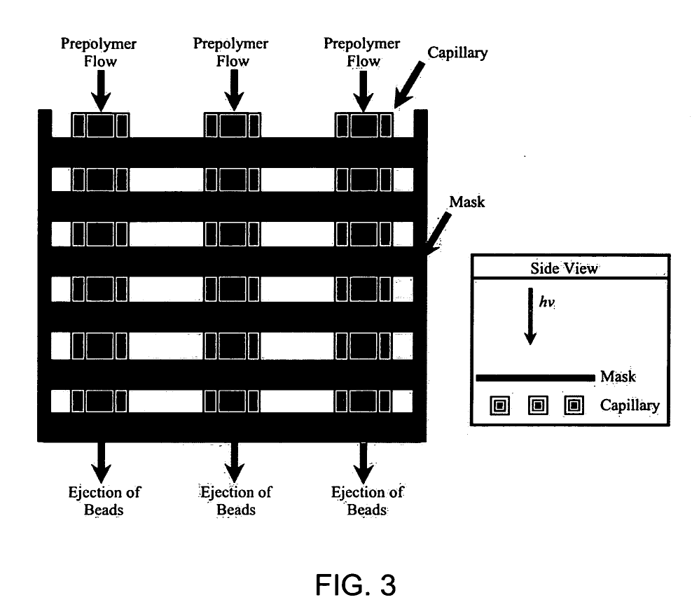

The microstructures of the invention can be made by a number of different chain-growth polymerization methods (e.g., PIPS, block copolymers) using a solid support having at least one confined space (e.g., 1, 2, 3, 4, 5, 10, 50, 1000 or more) that defines the geometry of the microstructures (e.g., molds, capillaries, and microchannels). In preferred methods of the invention, non-spherical polymeric microstructures are formed in a capillary (FIG. 2, FIG. 3) or in a microchannel (e.g., a microfluidics device having one or more channels with at least one dimension less than 1 mm, FIG. 1) to define the size and shape of two dimesions of the microcarrier (e.g., resulting in a fiber). A mask (FIG. 3) is used in conjunction with the capillary or microchannel to determine the size and shape of the third dimension of the microstructure (e.g., resulting in non-fiber shapes such as cubes, spheres, pyramids, etc).

PIPS is preferred for use in methods of the invention because this method of chain-growth polymerization allows for the creation of a polymeric microstructure with inter-connected porosity and nano-surface texture using a large number of possible monomers (see G. R. Yandek and T. Kyu, Macromol. Theory Simul. 14:312-324, 2005; Yu et al., Journal of Polymer Science, part A, 40:755-769, 2002). This large variety of possible monomers is due to the fact that in a PIPS system, a binary system of solvents can be used to dissolve the monomers; as long as the two solvents are inter-soluble then they can be used to dissolve the monomers to be polymerized. Therefore, almost any monomer, including hydrophilic monomers (e.g., 2-Hydroxyethyl methacrylate) which are generally not suitable for standard polymerization in aqueous suspension, may be used to form a PIPS polymer. When utilizing a PIPS system, any number of suitable chain-growth polymerization methods can be used, including free-radical, anionic, and cationic polymerization, etc. These methods are more efficient than the currently used suspension polymerized bead systems because hydrophobic and hydrophilic monomers are mixed in the same prepolymer solution leading to a one-step polymerization (Svec et al., 28:34-48, 1999).

In a typical method of the invention, a first step in making a polymeric microstructure is the selection of the prepolymer solution components. A polymeric microstructure made according to methods of the invention is a polymer made from a prepolymer solution containing at least one but typically a mixture of monomers (monofunctional, difunctional, and/or trifunctional), a mixture of solvents (allowing the monomers to be soluble and the polymer to be insoluble), and a photoinitiator (−1-5 wt % of monomer). A monomer suitable for use in the invention is one that has a group that can be attacked by a free-radical (e.g. acrylate or vinyl). Many different monomers exist that can be used in methods of the invention to synthesize microstructures including monofunctional, difunctional, and trifunctional monomers. Examples of monofunctional monomers include ethylene glycol-based monomers such as 2-Hydroxyethyl methacrylate (CH₂═C(CH₃)COOCH₂CH₂OH), Di(ethylene glycol) 2-ethylhexyl ether acrylate (H₂C═CHCO₂CH₂CH₂OCH₂CH₂OCH₂CH(C₂H₅)(CH₂)₃CH₃), and 2-Hydroxyethyl acrylate (CH₂═CHCOOCH₂CH₂OH); amine based-monomers such as 2-(Dimethylamino)ethyl methacrylate (CH₂═C(CH₃)COOCH₂CH₂N(CH₃)₂), and 2-(Diethylamino)ethyl methacrylate (H₂C═C(CH₃)CO₂CH₂CH₂N(C₂H₅)₂); and charged/responsive monomers such as Allylamine (CH₂═CHCH₂NH₂), 2-acrylamido-2-methylpropane sulfonic acid sodium salt (CH₂═CHCONHC(CH₃)₂CH₂SO₃H), Acrylic Acid (CH₂═CHCOOH), N,N-Dimethylacrylamide (CH₂═CHCON(CH₃)₂), and N-isopropylacrylamide (H₂C═CHCONHCH(CH₃)₂).

Examples of difunctional monomers include ethylene glycol-based monomers such as Di(ethylene glycol) diacrylate ((H₂C═CHCO₂CH₂CH₂)₂O), Ethylene glycol dimethacrylate (CH₂═C(CH₃)COOCH₂CH₂OCOC(CH₃)═CH₂), Tetraethylene glycol dimethacrylate (O[CH₂CH₂OCH₂CH₂OCOC(CH₃)═CH₂]₂), Triethylene glycol dimethacrylate (CH₂═C(CH₃)COO(CH₂CH₂O)₃COC(CH₃)═CH₂), and Tetra(ethylene glycol) diacrylate ((H₂C═CHCO₂CH₂CH₂OCH₂CH₂)₂O).

Trifunctional monomers such as Trimethylolpropane Triacrylate ((H₂C═CHCO₂CH₂)₃CC₂H₅) can also be used in methods of the invention. For a description of monomers that may be used in chain-growth polymerization techniques, see, for example, Polymer Chemistry, An Introduction, by Malcolm P. Stevens, 3^{rd} ed., Oxford University Press, NY, 1999.

Once the prepolymer mixture is created, a photoinitiator is added (usually 1-5 monomer wt %). For use as a photoiniator, a wide variety of photolabile compounds are available, including benzoin, benzil, 2,2′-azobisisobutyronitrile (AIBN), and disulfides (see, e.g., J.-P. Fouassier, Photoiniation, Photopolymerization, and Photocuring, Hanser, Cincinnati, 1995; and Fisher et al., Annu. Rev. Mater. Res. 31:171-181, 2001). Additional photoinitiators include Irgacure 184, Irgacure 651, Irgacure 819, Irgacure 1173, Irgacure 2022, Irgacure 2100, and Darocur, all commercially available from CIBA (Basel, Switzerland). After, the photoinitiator is added, the prepolymer mixture is flowed into the solid support having a confined geometry (e.g., microchannel, capillary) using either a pressure-driven flow (e.g., syringe pump, HPLC pump, etc.) or Electroosmotic Flow (EOF) (Gad-el-Hak, M., ed., The MEMS Handbook, 2001, CRC Press: Boca Raton, Fla.). If a fiber is to be formed, the prepolymer mixture is then exposed to irradiation (e.g., WV light) to initiate chain-growth polymerization.

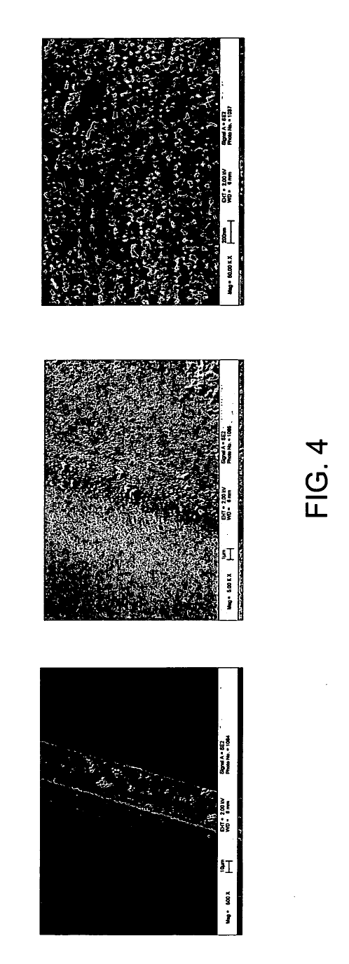

To produce cubical microstructures, for example, rather than a fiber, a mask is used in conjunction with the solid support. A mask is a device containing at least one portion that transmits at least one type of radiation (e.g., ultraviolet, infrared, visible light) and at least one portion that blocks transmission of the radiation. The mask allows only discrete portions of a prepolymer mixture in a capillary/microchannel to be exposed to polymerizing radiation. A mask can therefore be used to define one or more of the dimensions of a microstructure to be formed. Without a mask, continuous fibers are formed by these methods. Polymeric fibers were synthesized (FIG. 4, Example 1) using the square ID fused quartz capillary (cross-section of 50 μm×50 μm) shown in FIG. 2A and no mask (which constrains the polymer synthesis in the third dimension).

A typical mask for producing cubical microstructures consists of arrays of lines of widths between 50 μm and 100 μm. These three dimensions of constraint expose a 50 μm×50 μm×50 μm volume to UV light. A mask used in methods of the invention may be constructed by many methods. For example, the mask can be created in a thin gauge metal by a laser (Nd:YAG) cutting process; the molten metal ejected from the desired area using a high-pressure gas jet. A typical mask feature (hole, line, etc.) size using this method is between 50 μm and 100 μm and is dependent on material, material thickness, beam focus, and gas pressure (material removal rate).

To produce cubical microstructures using a capillary, polymerization is performed in a square ID fused quartz capillary (FIG. 2) using a mask so that the synthesis of the porous polymer is constrained in two dimensions. The microfluidic synthesis of microstructures using microchannels is very similar to the synthesis in capillaries, but instead of having individual capillaries, microfluidic synthesis typically involves an array of etched microchannels in a fused quartz wafer or glass slide. This allows for the synthesis of microstructures limited by solid support (e.g., chip, wafer, slide) size and channel width (>100 channels), instead of the limited number of capillaries that can be used in the capillary-based methods. The use of microfluidic channels also allows for complex fibers to be formed (e.g. core-sheath fibers). The first level of increased complexity is in the creation of a 2D focusing chip (two channels impinging on a third), which allows for focusing of the center channel. Formation of fibers in pure two-dimensional focusing creates a fiber that has an hourglass profile due to the friction on both the top and bottom surface of the channel. The next level of complexity that can be encompassed in microfluidic channel fiber synthesis is accomplished by making the impinging channels completely surround the central third channel, which allows the focusing to be three dimensional and removes the hourglass artifact. Modeling of water flow in a similar microfluidic channel setup has been undertaken by Chung (Chung et al., Microsystem Technologies-Micro- and Nanosystems Information Storage and Processing Systems 9(8): pages 525-533, 2003).

After the prepolymer mixture is irradiated by UV light, for example, the UV light impinges on a photoinitiator and causes the photoinitiator to decompose into a free-radical, thus initiating a chain-growth polymerizatoin. This resultant free-radical can attack any monomer that has a double bond (the initiation step of polymerization). This free-radical can then attack a new monomer, causing a chain-growth reaction (the propagation step of polymerization). During the polymerization propagation step, sub-particles are formed due to a particle nucleation (solubility-controlled reaction) of a polymer chain. These sub-particles are formed in a process where, as the polymer chain grows, the solubility parameter changes until it is no longer soluble in the system and the small particles nucleate. The size of these sub-particles is controlled by many parameters including the chain length at which the polymer becomes insoluble in the solvent mixture. The sub-particle coalescence is determined by the degree of cross-linking. Without using a cross-linker (a monomer having two or more functional groups), the individual sub-particles will almost completely coalesce, whereas if there is a high degree of crosslinker then there will be almost no coalescence of individual sub-particles. In cell culture applications where the polymeric microstructure is used as a microcarrier, the spaces between the sub-particles serve as inter-connected porosity for the microcarrier. The sub-particles also serve as a surface for the adhesion of cells, so that when the cells adhere to the surface they are adhering to a smoothly undulating surface (where the roughness is controlled by the size of the sub-particles).

The final step of this polymerization reaction occurs when the polymer chain is terminated by either disproportionation or combination (coupling). Whether termination occurs by disproportionation or combination depends in large measure on monomer structure and in particular, on the structure of the chain-end radical (see A. M. North and D. Postlewaite, in Structure and Mechanism In Vinyl Polymerization, by T. Tsuruta and K. F. O'Driscoll, Eds., Marcel Dekker, New York, 1969; and Polymer Chemistry, Malcolm P. Stevens, 3^{rd} ed., Oxford University Press, New York, 1999). After the polymer chain is terminated, polymerization-induced shrinkage of the polymeric microstructure occurs, allowing it to be ejected from the confining geometry. When using the capillary-based synthesis assembly (square capillary bed, mask, and UV light source) shown in FIG. 3, due to polymerization induced shrinkage, the final polymerized polymeric microstructure is approximately 45 μm×45 μm×45 μm. Examples of preferred protocols for creating a polymeric microstructure in a capillary and in a microchannel are described in Examples 4 and 5, respectively. The initiation, propagation, and termination steps of a typical polymerization are shown below.

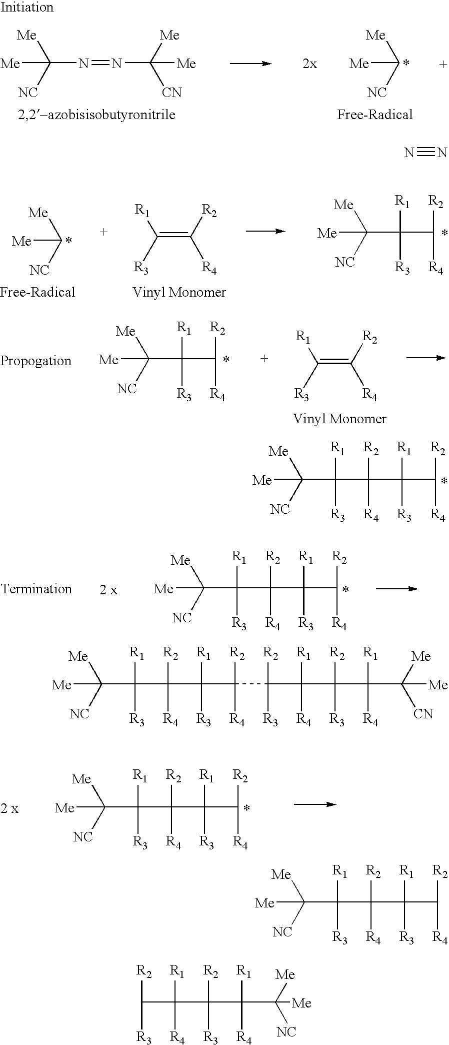

After polymerization is complete, the formed polymeric structures (microstructures) are ejected from the solid support. If being ejected from a capillary or microchannel, the polymeric microstructures are subjected to pressure from a syringe or vacuum pull. After the polymeric microstructures are ejected from the solid support having a confined geometry, they are purified by removing solvents and non-reacted monomers. This washing process can be performed by placing (soaking) the polymeric microstructures in a compatible solvent that can be easily removed (e.g., methanol soxhlet extraction for 24 hours). A soxhlet extracator utilizes a solvent (methanol-based) that is miscible in the polymer so that the polymer can swell and release both the solvents used in polymerization and unreacted monomers. After the soxhlet extraction, the soxhlet solvent is removed in a vacuum oven, and sterile water substituted as the carrier media. Solid supports used for synthesizing polymeric microstructures of the invention are preferably cleaned periodically with a solvent/acid mixture to prevent impurities from attaching on the interior surfaces of the solid suppport.

In addition to capillaries and microfluidic channels, microstructures described herein can be synthesized in a micromold. Examples of methods that can be used for the creation of micromolds include laser machining, traditional UV fabrication, and indentation. Micromolds utilized in the production of microstructures for cell culture applications (i.e., as microcarriers) can be one-sided molds or two-sided molds. By using a micromold, an array of over 1,000,000 (1000×1000) features can be produced on a 4-inch chip (e.g., fused quartz wafer, glass slide), thereby allowing for high productivity. Once the micromold is fabricated it is filled with a prepolymer and polymerized in the same manner as the capillary and microchannel- (e.g., microfluidic chip) based methods described above. After the microstructure is ejected from the micromold, it is purified as described above. In addition to chain-growth polymerization, step-growth polymerization techniques can also be used to synthesize polymeric microstructures in a micromold (see, Malcolm P. Stevens, 3^{rd} ed., Oxford University Press, New York, 1999).

In other embodiments, microstructures can be made using block copolymers (see, e.g., Jerome et al., Progr. Polym. Sci., (10), 87, 1994; Polymer Chemistry, Malcolm P. Stevens, 3^{rd} ed., Oxford University Press, New York, 1999). Block copolymers can be formed by using macromers with reactive (by free-radical polymerization) end-caps to form a phase separating system (e.g., cylindrical, spherical, lamella, and gyroid morphology). Two types of low molecular weight polymers can be end-capped with a group that can be attacked by a free-radical initiator. If this is a diblock (gyroid morphology) copolymer and one of the blocks is degradable, then that degradable block can be removed by hydrolysis, radiation, etc. This produces a nano-porous structure similar to that produced by PIPS. In all embodiments, regardless of the polymerization method or system used, the geometry, porosity, composition, and surface characteristics (e.g., topology, texture) of the resultant microstructures can be controlled.

After synthesis, the polymeric microstructures can be characterized for quality control. Techniques for characterizing the microstructures include microscopy, Fourier Transform Infrared (FTIR) spectroscopy, and cell culture demonstrations. Physical dimensions, shape, and other surface features can be examined using optical, confocal, and SEM, while FTIR ensures that the surfaces are clean of excess organic materials. When microstructures are to be used in cell culture applications, chemical analysis using FTIR can be used to determine if the polymer microstructure has been thoroughly cleaned and is ready for contact with cells. Any residual organic material can be identified and characterized using this technique. To assess cellular adhesion and viability of cells adhered to the microstructures, optical microscopy in the visible mode cam be used. The fluorescent mode of OM can be used to view actin and mitochondrial staining in the cells. In both modes, digital images can be captured from the microscope for analysis of cellular adhesion area using standard image analysis software. The same fluorescently labeled cells can also be studied using a confocal microscope. The confocal scanning images are used to construct a three-dimensional picture that provides an accurate image of cellular adhesion to the microstructures. SEM is used to characterize microstructure size and phase separation (particle/particle aggregate) sizes in the PIPS. To perform SEM, microstructures are oven dried and prepared according to standard SEM procedures. Microstructure dimensions, phase separation sizes, and general surface characteristics are measured using digitized SEM images. AFM imaging can also be used to characterize microstructures. AFM imaging of one microstructure sample synthesized using methods described herein showed a pristine molded surface, which was shown to be smoothly undulating with a roughness of 5-7 nm.

Using the methods described herein to produce polymeric microstructures, the solubility of the microstructure is controlled by the choice of monomers/macromers and solvent combinations used in the polymerization reaction. In a typical polymerization, as the polymer chain grows, the solubility changes. For example, if Ethylene Glycol Dimethacrylate (EGDMA) is polymerized, the solubility parameter changes from 19.3 J^{1/2}cm^{-3/2} (monomer) to 20.9 J^{1/2}cm^{-3/2} (2 units) to 21.5 J^{1/2}cm^{-3/2} (3 units) to 22.0 J^{1/2}cm^{-3/2} (6 units) to 22.3 J^{1/2}cm^{-3/2} (11 units) to a fully polymerized EGDMA at 22.5 J^{1/2}cm^{-3/2}. If the EGDMA is placed in a 50:50 solvent mixture of Methanol and Hexane, the solvent mixture's solubility parameter will be 19.7. The EGDMA monomer is soluble in this solvent mixture, but as the polymer chain grows, it becomes insoluble and starts to nucleate polymer particles. This control of the solubility parameter results in a predictable surface texture on the microstructure.

These methods also provide a flexible polymer chemistry due to the fact that both hydrophobic and hydrophilic monomers can be polymerized, and this flexible chemistry allows for control of cellular adhesion properties and cell spreading/growth. For example, to promote adhesion of cells to microstructures, it is preferred that such microstructures be produced from reagents that result in a positively-charged surface. Electrostatic interaction between the cell (negatively charged) and the polymer surface (positively charged) will cause a non-specific binding of the cell to the surface of the polymer. Ionizable groups can also be used to provide a charged surface (positive or negative) to the microstructure dependent upon the application in which the microstructure is used.

### Microstructures Having Pre-Determined Geometries and Surface Characteristics

The invention provides polymeric microstructures having a pre-determined geometry and pre-determined surface characteristics. Microstructures are synthesized to have pre-determined geometric, chemical, and topographical characteristics including, for example, size, curvature, shape, porosity, texture, and functional group attachment. Microstructures described herein can be any shape including spheres and regular and irregular polyhedrons (e.g., cubes, prisms, pyramids, tetrahedrons, octahedrons, dodecahedrons, rhombic dodecahedrons, trapezohedra, icosadedrons, stellated polyhedrons). For use as microcarriers in tissue culture, they are typically either cubes or rectangular prisms having rounded corners to prevent damage to cells adhered thereto.

Generally, the microstructures are between about 30 and 500 microns (e.g., 25, 30, 40, 50, 60, 70, 80, 90, 100, 200, 300, 400, 500, or 600 microns) in size, although they may be of other sizes (e.g., between about 1-1000 microns) depending on the particular application contemplated. Compared to currently available spherical microstructures which typically are about 130 μm in diameter and have a surface area of 53,093 μm², a volume of 1.15×10⁶ μm³, and a surface area to volume ratio of 0.046 μm⁻¹, the surface area of a typical cuboidal microstructure (50 μm×50 μm×50 μm) of the invention is 15,000 μm², the volume is 125,000 μm³, and the surface area to volume ratio is 0.12 μm⁻¹. Polymeric fibers synthesized by methods described herein typically have a dimension that is much greater than the other two dimension, x₁≈x₂<<x₃ (e.g., x₁=x₂=50 microns and x₃=1000 microns).

Polymeric microstructures described herein are not limited to these dimensions, however, and can have a surface area in the range of about 5400 μm² to about 1.5×10⁶ μm² (e.g., about 6000 to about 1.0×10⁶, about 7000 to about 1.5×10⁵, about 8000 to about 1.0×10⁵, about 9000 to about 1.5×10⁴, about 10,000 to about 14,000, about 11,000 to about 13,000, about 11,500 to about 12,500, about 12,000 to about 12,100 μm² etc.), a volume in the range of about 27000 μm³ to about 1.25×10⁸ μm³ (e.g., about 27,000 to about 1.25×10⁸, about 30,000 to about 1.0×10⁸, about 35,000 to about 1.5×10⁷, about 40,000 to about 1.0×10⁷, about 45,000 to about 1.5×10⁶, about 50,000 to about 1.0×10⁶, about 55,000 to about 1.5×10⁵, about 60,000 to about 1.0×10⁵, about 65,000 to about 90,000, about 70,000 to about 80,000, about 75,000 to about 79,000 μm³, etc.), and a surface area to volume ratio in the range of about 0.2 μm⁻¹ to about 0.012 μm⁻¹ (e.g., about 0.13 to about 0.018, about 0.09 to about 0.027, about 0.06 to about 0.04, about 0.055 to about 0.05 μm⁻¹, etc.).

In some embodiments, microstructures of the invention are preferably porous. In cell culture applications, this porosity allows for nutrient and waste diffusion through a cellular/microstructure aggregate. Porous microstructures also provide a large surface area for bonding (reversible or irreversible) of functional groups or biologically active molecules. The connected porosity of the microstructures further allows for water access to the inner surface of the microstructures for engineered degradation, and for the creation of a surface texture that can modify cell adhesion and cytoskeleton development.

A polymeric microstructure made according to methods of the invention is a polymer made from a prepolymer solution containing at least one but typically a mixture of monomers (monofunctional, difunctional, and/or trifunctional), a mixture of solvents (allowing the monomers to be soluble and the polymer to be insoluble), and a photoinitiator (˜1-5 wt % of monomer).

In some embodiments, the microstructures described herein are biodegradable, so that all synthetic material is eliminated from the bioreactor-based tissue model. A bioreactor-based tissue model is tissue grown in a bioreactor that has been grown in biodegradable matrix or on microcarriers that has form inter-microcarrier cell-cell bridges. The tissue grown in the bioreactor could be developed from any type of cells (e.g. cardiac cells, cancer cells, etc.). Some common linkages seen in these biodegradable polymers include ester, amide, and anhydride, as shown in the ester (top left), amide (top right), and anhydride (bottom) hydrolysis reactions below (Michael B. Smith and Jerry March, Advanced Organic Chemistry, 2001).

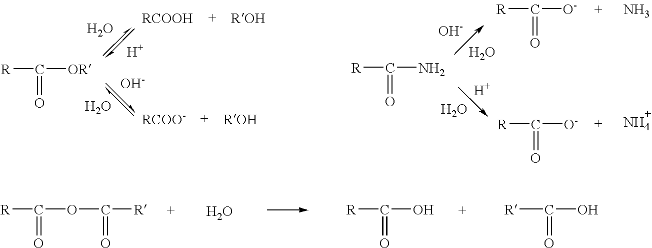

The microstructures made according to methods of the invention are synthesized to have particular surface characteristics. As described above, control of the solubility parameter results in a predictable surface texture on the microstructure (see FIG. 4). Microstructures can be composed of biocompatible or even biomimicking polymers, for example. For use in cell culture applications, the polymeric microstructures are preferably biocompatible and also have an appropriate surface energy, since cells prefer to adhere to hydrophilic surfaces. A pre-determined surface energy can be obtained through the proper choice of monomers which are allowed in a greater variety with a PIPS system.

In addition to the surface characteristics described above, microstructures of the invention can be bound by or attached to a variety of functional groups (e.g., peptides, nutrients), the microstructures providing an excellent surface for absorption or covalent bonding of such functional groups. Any suitable naturally occurring or synthetically generated biomolecule (e.g., RGD sequence, glucose, etc.) can be grafted or immobilized on the surface of the microstructure. In one embodiment of the invention, RGD sequences, an adhesion promoter peptide sequence, are bonded to a microstructure. Because this peptide sequence acts as a cellular adhesion site, microstructures having this sequence covalently immobilized provide increased cellular adhesion area when used as a microcarrier in cell culture. Some common methods for the modification of a biomaterial surface with RGD are reviewed in Hersel et. al. (Hersel et al., Biomaterials, 24: p. 4385-4415, 2003). As another example, a nutrient molecule such as glucose can be bonded to a microstructure. The ability to attach glucose to a cell culture microstructure with a degradable linkage (e.g. ester, amide, anhydride) allows for time-delayed access to a vital nutrient molecule. This is useful when a cell aggregate forms, since nutrient access at the center of the cell aggregate becomes diffusion-limited.

In addition to glucose and RGD, many other biologically active molecules can be immobilized or covalently bonded to the surface of the microstructure (see, for example, Chapter 2 of Ratner, B. D., A. S. Hoffman, F. J. Schoen, and J. E. Lemons, Biomaterials Science. 2004, Oxford, UK: Elsevier Inc.). Examples of these molecules include proteins/peptides (e.g., enzymes, antibodies, antigens, “blocking” proteins, and cell adhesion molecules), saccharides (e.g., sugars, oligosaccharides, and polysaccharides), lipids (e.g., fatty acids, phospholipids, and glycolipids), drugs (e.g., antithrombogenic agents, anticancer agents, antibiotics, contraceptives, drug antagonists, peptides, and protein drugs), ligands (e.g., hormone receptors, cell surface receptors, avidin, and biotin), nucleic acids, nucleotides, single- and double-stranded DNA, and RNA (e.g., antisense oligonucleotides). In addition, the pores of the microstructure can be filled with a mixture of a biodegradable polymer (e.g., polyanhydride) and a nutrient molecule for controlled delivery of nutrient molecules. A number of biodegradable polymers are known in the art, including polyglycolic acid (PGA), polylactic acid (PLA), lactic acid-glycolic acid copolymer (PLGA), poly-ε-caprolactone (PCL), polyamino acid, polyanhydride, polyorthoester, and copolymers thereof. See U.S. Pat. No. 6,562,374. Incorporating these components into the microstructure reduces the appearance of necrotic cores in large cell aggregates. A necrotic core is formed when a cell/microcarrier aggregate becomes so large that diffusion of nutrients and waste products can not be moved in and out of the center of the aggregate.

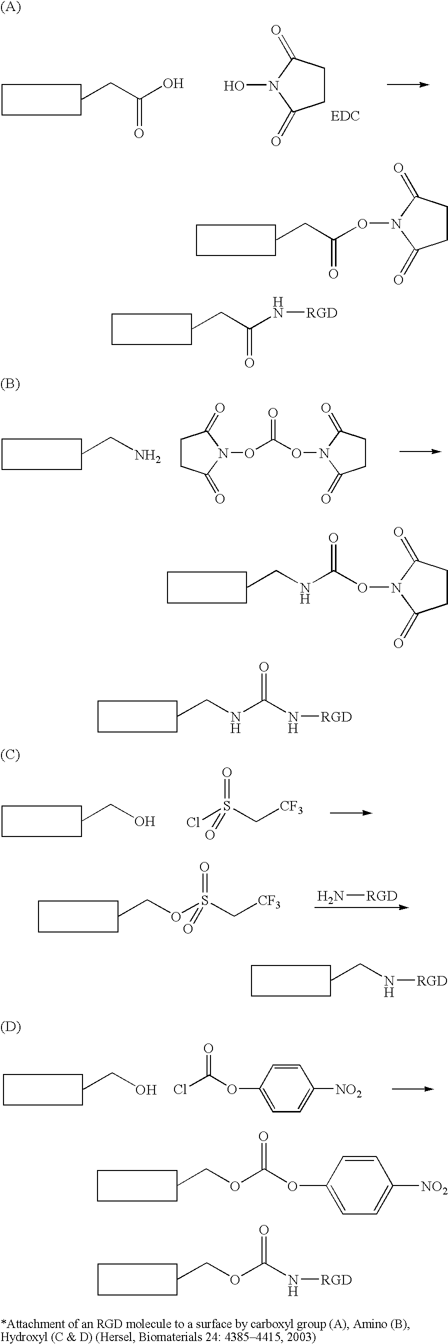

### Applications of Polymeric Microstructures

The microstructures of the invention can find use in a number of applications. Because of the geometries and surface characteristics described above, many of the microstructures described herein are especially useful for culturing cells in a bioreactor, particularly cell lines that are fragile and/or anchorage-dependent. Microstructures of the invention preferred for cell culture applications feature a high surface area to volume ratio. The increase in growth surface or adhesion area provided by the dimensions of the microstructures described herein over currently available spherical microstructures results in cells growing in a flatter manner (rather than rounding or clumping up), which reduces the shear stresses on the cell in a bioreactor culture environment and increases the metabolic activity of the cell. This increase in growth surface/adhesion area also reduces the occurrence of necrotic cores in bioreactor cell growth which result from insufficient nutrients reaching the interior of an aggregate of cells and from cellular waste not sufficiently passing from the interior of the aggregate to the surface of the aggregate. Because of the increase in growth surface/adhesion area, the distance nutrients must travel to reach the interior of an aggregate of cells, and the distance waste must travel from the interior of an aggregate of cells to the surface of the aggregate is reduced, thereby reducing the occurrence of necrotic cores.

For use in cell culture applications, especially preferred microstructures are thin with large flat surfaces to facilitate cell adhesion and growth while minimizing displaced volume. The microstructures for cell culture applications are also preferably porous to allow for nutrient and waste diffusion through a cellular/microstructure aggregate. Porous microstructures also provide a large surface area for bonding (reversible or irreversible) of functional groups or biologically active molecules

For use in tissue engineering applications, preferred microstructures have a geometry that provides for a low-packing efficiency. This low packing efficiency allows for a bimodal pore distribution on the surface and micropores between particles. A typical geometry for use in tissue engineering applications is the shape of a cross.

In one example of tissue engineering, the polymeric microstructures of the invention can be used in bone tissue engineering. Current methods of bone tissue engineering utilize large filler material or in-situ polymerized material. Some of the drawbacks of the in-situ polymerized material are heat generation due to polymerization, non-uniform porosity, non-uniform composition, and incomplete filling of void spaces. A bone tissue engineering microcarrier (BTEM) made by methods of the invention has a particular, three-dimensional geometry providing an improved material for forming bone tissue because it has properties in between the in-situ polymerized materials and the large filler materials. The BTEM display the mechanical interlocking and space-filling properties of the in-situ polymerized material and the ease of utilization seen with the larger filler material. A preferred BTEM made by methods described herein is composed of a biodegradable component (e.g., polyanhydride) and a temperature- or pH-actuated hydrogel segment. This combination of components allows the BTEM to expand upon implantation and then slowly degrade during bone ingrowth. Other components can be added to this polymer system either during or after polymerization, such as hydroxyapatite, to enhance bone growth.

FIG. 1 shows a microfluidic chip that can be used for the production of BTEM crosses. By utilizing a cross geometry in which a plurality of microstructures are packed together, large pores (e.g., approximately 100 μm) are created by the low packing efficiencies. This space filling procedure allows for independent control of the surface texture of the crosses and cellular porosity (e.g., inter-cross pores). Large porosity is preferred for bone cells and nano-porosity and nanotexturing for cellular adhesion.

By performing surface modification involving attachment of a labile linkage to a glycosaminoglycan (GAG) (See Chamberlain, L J, I V Yannas, H P Hsu, and M. Spector, Tissue Engineering 3 (4) 353-362, 1997), the polymeric microstructures synthesized as microstructures as described above can be used for the regeneration of cartilage tissue. These structures are useful for cartilage tissue regeneration because they have an interlocking geometry (e.g. cube, star, cross) and because they can be injected into the articulating joint capsule with minimal disruption of the joint capsule due to their size (about 50-100 μm). Once these polymeric microstructures are injected into the capsule, the temperature or pH-sensitive hydrogel component of the structures expands and interlocks to fill the cavity.

### EXAMPLES

The present invention is further illustrated by the following specific examples. The examples are provided for illustration only and are not to be construed as limiting the scope or content of the invention in any way.

### Example 1

### Synthesis of Fibers

The fibers shown in FIG. 4 were made in a four-fold axial symmetric capillary (square cross-section as seen in FIG. 2) using a poly(Trimethylolpropane Triacrylate-co-ethylene dimethacrylate) System. The prepolymer solution was flowed into the UV-exposed region of a capillary at a low, constant flow rate. The pre-polymer system used in this synthesis was a PIPS solution so, at a point determined by the thermodynamics of the system, there was a nucleation and growth phase separation that occurred. This phase-separation created the porous structured fibers seen in FIG. 4. This synthesis can also be accomplished in the same manner using a microfluidic chip. The fiber seen in the image can be seen to be composed of partially coalesced nano-particles. The surfaces of these fibers show a roughly uniform level of surface roughness and a high level of porosity. The protocol listed below was conducted 25° C. and 1 atm. The polymer flow was induced using a low-flow rate syringe pump.
1. Created the prepolymer that was used to synthesize poly(Ethylene Glycol Dimethacrylate-co-Trimethylolpropane Triacrylate) square cross-sectioned fibers. The poly(Ethylene Glycol Dimethacrylate-co-Trimethylolpropane Triacrylate) system utilized 0.36 μL of EGDMA (δ_{D}=17.2 J^{1/2}cm^{-3/2}, δ_{P}=0.2 J^{1/2}cm^{-3/2}, δ_{H}=8.8 J^{1/2}cm^{-3/2}, δ=19.3 J^{1/2}cm^{-3/2}), 0.24 μL of Triethylolpropane Triacrylate (TMPTA, δ_{D}=17.01 J^{1/2}cm^{-3/2}, δ_{P}=0.15 J^{1/2}cm^{-3/2}, δ_{H}=9.01 J^{1/2}cm^{-3/2}) 6.5 mg of 2,2′-azobisisobutyronitrile (AIBN), and 0.9 μL of a porogenic solvent. The porogenic solvent mixture was composed of dimethylforamide (DMF, δ_{D}=17.4 J^{1/2}cm^{-3/2}, δ_{P}=13.7 J^{1/2}cm^{-3/2}, δ_{H}=11.3 J^{1/2}cm^{-3/2}, δ24.9 J^{1/2}cm^{-3/2}) and tetrahydrofuran (THF, δ_{D}=16.8 J^{1/2}cm^{-3/2}, δ_{P}=5.7 J^{1/2}cm^{-3/2}, δ_{H}=8.0 J^{1/2}cm^{-3/2}, δ=19.5 J^{1/2}cm^{-3/2}). In this system the monomers were EGDMA and TMPTA, the solvents were DMF and THF, and AIBN was used as a free-radical initiator.
2. The 50 micron square capillary was cleaned with THF and 5% Sulfuric acid
3. Flowed THF to remove the cleaning solution
4. Flowed the prepolymer solution created in step 1 into the capillary
5. Exposed the capillary to UV light in a UV oven for 30 minutes to allow for polymerization of the fiber.
6. Flowed new prepolymer solution into the capillary. This step caused the already synthesized fiber to be ejected from the capillary.

Phase separation occurs because the polymerized EGDMA shifts to a higher solubility parameter of δ=22.5 J^{1/2}cm^{-3/2} (δ_{D}=20 J^{1/2}cm^{-3/2}, δ_{P}=0.2 J^{1/2}cm^{-3/2}, δ_{H}=10.4 J^{1/2}cm^{-3/2}) and the polymerized TMPTA shifts to a higher solubility parameter of δ=29.0 J^{1/2}cm^{-3/2} (δ_{D}=25.9 J^{1/2}cm^{-3/2}, δ_{P}=0.311 J^{1/2}cm^{-3/2}, δ_{H}=13.0 J^{1/2}cm^{-3/2}). Since the polymer being created is a random copolymer of these two monomers, the actual solubility of the polymer created will be somewhere in between the pEGDMA and the pTMPTA. It can be seen from the solubility parameters listed in step one and above for the polymerized form of the monomers that the monomers are initially soluble in the solvents used and then as the polymerization occurs the polymer is ejected from solution by either a nucleation growth or spinodal decomposition. SEM images were obtained of the square fibers produced (FIG. 4). The fibers show a highly porous morphology with particle sizes of about 30-50 nm. The fiber diameter is 42 microns, indicating that there is a 16% polymerization induced contraction. The fiber surface is flat, so the large particles seen on the surface of some of the images are dirt contaminates.

### Example 2

### Another Example of a Prepolymer System

Another prepolyer system that can be utilized in the same manner as in Example 1 is composed of 0.96 g of Ethylene Glycol Dimethacrylate (EGDMA, δ_{D}=17.2 J^{1/2} cm^{-3/2}, δ_{P}=0.2 J^{1/2}cm^{-3/2}, δ_{H}=8.8 J^{1/2}cm^{-3/2}, δ=19.3 J^{1/2}cm^{-3/2}), 1.301 g of Hydroxyethylmethacrylate (HEMA, δ_{D}=17.8 J^{1/2}cm^{-3/2}, δ_{P}=0.4 J^{1/2}cm^{-3/2}, δ_{H}=14.8 J^{1/2}cm^{-3/2}, δ=23.2 J^{1/2}cm^{-3/2}), 24 mg of AIBN, and 3.6 g of a porogenic solvent in a UV-initiated polymerization reaction. The solvent system used is Methanol (MeOH, δ_{D}=15.2 J^{1/2}cm^{-3/2}, δ_{P}=12.3 J^{1/2}cm^{-3/2}, δ_{H}=22.3 J^{1/2}cm^{-3/2}, δ=29.2-29.7 J^{1/2}cm^{-3/2}) and Hexane (δ_{D}=14.8 J^{1/2}cm^{-3/2}, δ_{P}=0 J^{1/2}cm^{-3/2}, δ_{H}=0 J^{1/2}cm^{-3/2}, δ=14.8-14.9 J^{1/2}cm^{-3/2}). In the pure methanol solvent system the median pore size is 54 nm, in the MeOH/EtOH (50/50) system the median pore size is 51 nm, and in the MeOH/Hexane (50/50) system the median pore size is 7959 nm. Polymerized EGDMA shifts to a higher solubility parameter δ=22.5 J^{1/2}cm^{-3/2} (δ_{D}=20 J^{1/2}cm^{-3/2}, δ_{P}=0.2 J^{1/2}cm^{-3/2}, δ_{H}=10.4 J^{1/2}cm^{-3/2}). Polymerized HEMA shifts to a higher solubility parameter δ=34.6 J^{1/2}cm^{-3/2} (δ_{D}=26.9 J^{1/2}cm^{-3/2}, δ_{P}=0.8 J^{1/2}cm^{-3/2}, δ_{H}=21.8 J^{1/2}cm^{-3/2}). As can be seen from the increase in the solubility parameter from monomer (HEMA's δ=23.2 J^{1/2}cm^{-3/2} and EGDMA's δ=19.3 J^{1/2}cm^{-3/2}) to polymer (pHEMA's δ=34.6 J^{1/2}cm^{-3/2} and pEGDMA's δ=22.5 J^{1/2}cm^{-3/2}), it is expected that these monomers would be soluble and then come out of a solution when they polymerize in a solution of Methanol (δ=29.2-29.7 J^{1/2}cm^{-3/2}) and Hexane (δ=14.8-14.9 J^{1/2}cm^{-3/2}). See Yu et al., Journal of Polymer Science: Part A, 40:755-769, 2002.

### Example 3

### Steps in a Method of Micromold Operation

This example embodies one of the three preferred methods of creating microcarriers. Micromolding provides the ability to create thousands of microcarriers in one synthesis cycle. This method of fabrication utilizes two microfabricated fused quartz wafers as molds for UV synthesis of the desired cubical microcarriers. This method includes the following steps:

1) Fabrication (UV lithography, laser processing, etc.) of two replicate surfaces.

2) When the surfaces are glass, the surfaces are treated with a material (e.g., flourosilane) that deactivates the surface, so the polymer does not adhere to the surface of the mold.

3) The molds are filled with the prepolymer solution

a. With Interconnecting Channels

b. The chips are aligned and the assembly is closed

i. The prepolymer solution is degassed prior to injection into the assembly

ii. The assembly is then filled with a prepolymer from a central port

iii. Without Interconnecting Channels

c. The chips are placed in a prepolymer solution

i. The system is placed under vacuum and then backfilled with nitrogen (degassing the prepolymer system).

ii. The chips and solution are agitated (ultrasound, stirring, etc.) to remove air bubbles on chip surface

iii. The chips are brought together and aligned.

4) The assembly is placed under pressure to remove the thin film of prepolymer between the two chips.

5) The assembly is irradiated with UV light to induce a PIPS reaction (this can be done with a mask or with out a mask).

### Example 4

### Steps in a Method of Capillary Bed Operation

This method utilizes a similar protocol to Example 1, but a mask is added to control exposure of the prepolymer to UV light in the third dimension. The exposure regions in the capillary synthesis are an array of 50 micron×50 micron×50 micron cubical regions inside the capillary. Upon polymerization, these UV exposed regions create the cubical microcarriers. This method includes the following steps:

1) Fabrication of UV mask out of a thin gauge metal using laser machining. This mask is composed of an array of 50 micron thick lines that only let UV light pass through mask in these laser machined lines to expose the prepolymer solution in the capillaries.

2) The assembly is treated with a chemical that deactivates the surface (e.g., flourosilane), so the polymer does not adhere to the surface of the assembly.

3) The prepolymer solution is degassed.

4) The capillaries are filled with the prepolymer solution (positive pressure, vacuum, electroosmotic flow, etc.)

5) UV radiation of the assembly to induce a chain-growth polymerization that leads to phase separation at a critical chain length.

6) Application of a liquid force to cause the fabricated three-dimensional structures to leave their confined environment. The polymer will undergo shrinkage during polymerization, so this process does not require large forces.

7) During the ejection of the previously fabricated microstructure the capillaries refills with prepolymer.

### Example 5

### Steps in a Method of Microfluidic Channel Operation

This method is similar to the method of Example 4, but instead of using an array of capillaries and a mask, the whole assembly (microfluidic channel arrays and mask) is assembled on one chip. The exposure regions in the microfluidic chip synthesis are an array of 50 micron×50 micron×50 micron cubical regions inside the capillary. Upon polymerization, these UV exposed regions create the cubical microcarriers. This method includes the following steps:

1) UV lithography, micromachining, laser ablation, dry reactive ion etching, or other methods are used to fabricate the component wafers.

2) The component wafers are bonded together.

3) A region of the assembly is selectively made UV transparent by making part of the assembly opaque with a deposited metal or mask.

4) The assembly is treated with a chemical that deactivates the surface (e.g. flourosilane), so the polymer does not adhere to the surface of the assembly.

5) The prepolymer solution is degassed.

6) The microchannels are filled with the prepolymer solution (positive pressure, vacuum, electroosmotic flow, etc.)

7) UV radiation of the assembly to induce PIPS reaction.

8) Application of a force to cause the fabricated three-dimensional structures to leave their confined environment. The polymer will undergo shrinkage during polymerization, so this process does not require large forces.

9) During the ejection of the previously fabricated microstructure the channel refills with prepolymer.

### Example 6

### Continuous Synthesis of Fibers

If a continuous fiber is desired, the syringe pump can be continually run at a slow rate through the UV oven. As long as the flow rate is slow enough for polymerization to reach greater than 70% conversion prior to leaving the capillary/microfluidic channel, then the fiber should have the mechanical integrity to stand on its own. This can be accomplished in either a capillary or a microfluidic chip. A method for producing a continuous fiber includes the following steps:

1) Follow steps 1-5 of Example 5

2) Maintain a constant slow flow of the reactive fluid

3) Expose a slit of the channel to UV light

### Other Embodiments

While the above description contains many specifics, these should not be construed as limitations on the scope of the invention, but rather as examples of preferred embodiments thereof. Many other variations are possible. For example, although the description of the invention focuses on synthesizing polymeric microstructures using chain-growth polymerization techniques, the invention could also be implemented using step-growth polymerization techniques or any other suitable techniques. Accordingly, the scope of the invention should be determined not by the embodiments illustrated, but by the appended claims and their legal equivalents.

What is claimed is:

1. A method for producing a polymeric microstructure having a pre-determined geometric shape, the method comprising the steps of:
(a) providing a solid support having at least one channel or cavity therein, the at least one channel or cavity defining at least a portion of the pre-determined geometric shape;
(b) applying a prepolymer mixture to the at least one channel or cavity;
(c) polymerizing the prepolymer mixture in the at least one channel or cavity to result in the formation of the polymeric microstructure having the pre-determined geometric shape, and
(d) removing the polymeric microstructure from the solid support.

2. The method of claim 1, wherein the solid support is selected from the group consisting of: a mold, a capillary, a slide, a pair of slides, and a device comprising at least one microfluidic channel.

3. The method of claim 1, wherein step (c) is performed by Polymerization Induced Phase Separation.

4. The method of claim 1, wherein step (c) is performed by polymerizing macromers to create block copolymers.

5. The method of claim 1, wherein step (c) comprises subjecting the prepolymer mixture to a sufficient amount of irradiation to polymerize the prepolymer mixture.

6. The method of claim 5, wherein the irradiation consists essentially of ultraviolet light.

7. The method of claim 5, further comprising using a mask to block the irradiation from at least one discrete portion of the prepolymer mixture in the at least one channel or capillary.

8. The method of claim 1, wherein step (d) comprises applying pressure to the polymeric microstructure within the at least one channel or cavity.

9. The method of claim 1, wherein the prepolymer mixture comprises at least one hydrophobic monomer and at least one hydrophilic monomer.

10. The method of claim 9, wherein the prepolymer mixture comprises Ethylene Glycol Dimethacrylate-co-Trimethylolpropane Triacrylate and Triethylolpropane Triacrylate.

11. A polymeric microstructure having a pre-determined geometry, the polymeric microstructure formed from the process comprising the steps of:
(a) providing a solid support having at least one channel or cavity therein, the at least one channel or cavity defining at least a portion of the pre-determined geometric shape;
(b) applying a prepolymer mixture to the at least one channel or cavity;
(c) polymerizing the prepolymer mixture in the at least one channel or cavity to result in the formation of the polymeric microstructure having the pre-determined geometric shape, and
(d) removing the polymeric microstructure from the solid support.

12. The polymeric microstructure of claim 11, wherein the size of the polymeric microstructure is between about 30 and 500 microns.

13. The polymeric microstructure of claim 11, wherein the shape of the polymeric microstructure is a regular polyhedron.

14. The polymeric microstructure of claim 11, wherein the shape of the polymeric microstructure is an irregular polyhedron.

15. The polymeric microstructure of claim 11, wherein the shape of the polymeric microstructure is selected from the group consisting of cube, prism, pyramid, sphere, tetrahedron, octahedron, dodecahedron, rhombic dodecahedron, trapezohedron, icosahedron, and stellated polyhedron.

16. The polymeric microstructure of claim 11, wherein the polymeric microstructure is a fiber.

17. The polymeric microstructure of claim 11, wherein the polymeric microstructure is porous.

18. The polymeric microstructure of claim 11, wherein the polymeric microstructure is linked to at least one functional group selected from the group consisting of: a peptide, a protein, an antibody, a saccharide, an organic molecule with a molecular weight of less than 10,000 daltons, and a nucleic acid.

19. The polymeric microstructure of claim 11, wherein the polymeric microstructure has a surface area in the range of about 5400 μm² to about 1.5×10⁶ μm², a volume in the range of about 27000 μm³ to about 1.25×10⁸ μm³, and a surface area to volume ratio in the range of about 0.2 μm⁻¹ to about 0.012 μm⁻¹.

20. The polymeric microstructure of claim 11, wherein the polymeric microstructure comprises a biodegradable polymer.
